(19) United States
(12) Patent Application Publication
(10) Pub. No.: US 2023/0149441 A1
(43) Pub. Date: May 18, 2023

(54) PHARMACEUTICAL COMPOSITION FOR TREATMENT OF ENVELOPED DNA OR RNA VIRUS INDUCED INFECTIONS AND DISORDERS

(71) Applicant: LOGICK ENERGETICS B.V., Leiden (NL)

(72) Inventors: Gerard KRIELEN, Leiden (NL); Clemens Waltherus Gerardus LÖWIK, Rotterdam (NL)

(21) Appl. No.: 17/995,823

(22) PCT Filed: Apr. 9, 2021
(86) PCT No.: PCT/EP2021/059349

§ 371 Date: Oct. 7, 2022

(30) Foreign Application Priority Data
Apr. 10, 2020 (NL) 2025335

(51) Int. Cl. A61K 31/737; A61K 31/405; A61K 31/4439

(52) U.S. Cl. CPC A61K 31/737; A61K 31/405; A61K 31/4439

(57) ABSTRACT

The invention relates to a composition, as well as a dosage, and a medicament for treatment of enveloped DNA or RNA virus induced infections and disorders, such as respiratory tract infection, and lung fibrosis. It has been found that the present composition limits the effects of virus infection on the human body, and contributes to the recovery thereof.

### FIELD OF THE INVENTION

The invention relates to a composition, as well as a dosage, and a medicament for treatment of enveloped DNA or RNA virus induced infections and disorders, such as respiratory tract infection, and lung fibrosis. It has been found that the present composition limits the effects of virus infection on the human body, and contributes to the recovery thereof.

### BACKGROUND OF THE INVENTION

The occurrence of virus infections is widely spread. A viral disease or infection, occurs when a living organism is invaded by pathogenic viruses, which cause infectious virus particles to attach to and enter susceptible cells in the organism. There are many types of viruses that may cause infection. Viruses may be “enveloped” or “non-enveloped”.

Virus infections may be transmitted from one organism to another, by various routes. Some viruses can only be transmitted within a organism species, such as from humans to humans, and some viruses may be transmitted from species to species.

Virus infections may occur throughout a body of an organism, or mainly at specific locations therein, such as in the lungs, in the gastroenteritis, and so on.

For treatment of these virus infections typically no adequate treatment is available. Often only symptoms of the infection can be mitigated, such as by assisting the lung function by an artificial respiration apparatus. Development of a medicament is a rather long process, typically involving at least months, and often more than a year. Often vaccination is possible in advance, if a medicament can be developed in advance, assuming a certain type of virus infection to occur. But vaccination for aggressive virus infections is seldom achieved.

WO 2004/093888 A recites methods for treating, preventing or managing virus infections in mammals using sulfated polysaccharides. More particularly, this invention relates to methods of treating, preventing or managing infection by viruses, particularly viral infections leading to or causing diseases. The invention involves the use of sulfated polysaccharides which are abundant, non-toxic and inexpensive and which are potent antivirals in vivo. WO 2009/027057 A1 recites the use of iota- and/or kappa-carrageenan for the manufacture of an antiviral pharmaceutical composition for the prophylaxis or treatment of a pathological condition or disease caused by or associated with an infection by a respiratory virus. The present invention further provides for the use of fucoidan, in particular of high molecular weight fucoidan, for the manufacture of an antiviral pharmaceutical composition for the prophylaxis or treatment of a pathological condition or disease caused by or associated with an infection by a respiratory virus. Amici et al. in ANTIVIRAL THERAPY—AN OFFICIAL PUBLICATION OF THE INTERNATIONAL SOCIETY FOR ANTIVIRAL RESEARCH, Vol. 11, No. 8, Jan. 2006, pp. 1021-1030, recites that Severe acute respiratory syndrome (SARS) is a newly emerging, highly transmissible and fatal disease caused by a previously unknown virus. Existing in non-identified animal reservoirs, this virus continues to represent a threat to humans because there is no effective specific antiviral therapy for virus infections. Objectives formulated are that starting from the observation that cyclopentenone cyclooxygenase (COX) metabolites are active against several RNA viruses, the effect of the COX inhibitor indomethacin is investigated on virus replication. WO 2005/013980 recites the use of indomethacin (INDO) and its derivatives and salts as antiviral drugs, since it was found that INDO is able to stimulate an antiviral defence response in cells attacked by viruses. This antiviral response has been found in the presence of INDO alone and/or in combination with other compounds, for instance with metals and metal-containing compounds, Prostanoids and antiviral drugs. In combination with these compounds INDO develops an unexpected as well as effective synergic antiviral action. Xu et al. in https://doi.org/10.1101/2020.04.01.017624 investigate effective antiviral agents to combat viral diseases. Previous studies found that indomethacin has the ability to inhibit the replication of several unrelated DNA and RNA viruses. WO 2011/100805 A1 recites anti-viral therapy and prophylaxis. Formulations and agents are provided which inhibit viruses of the Orthomyxoviridae family and ameliorate symptoms and conditions caused by viral infection. The present disclosure teaches the control of influenza virus infection and spread and amelioration of conditions caused thereby. The formulations and agents may be processed as medicaments or as health supplements for more general application such as in the form of consumer goods or consumer foods.

As treatment of virus infections are scarce or ineffective, there still is a need for improved pharmaceuticals and treatments which overcome drawbacks of the prior art.

### SUMMARY OF THE INVENTION

It is an objective of the invention to provide a composition that is able to treat enveloped DNA or RNA virus induced infections and disorders. The invention provides for a pharmaceutical composition for use in the treatment of enveloped DNA or RNA virus induced infections and disorders, comprising a first compound comprising at least one carbohydrate and containing more than one sulphate, and a second compound, different from the first compound, that can activate PPAR, preferably PPARγ. The first compound preferably comprises two or more sulphates, and is more preferably an oligo-, or poly-sulphate comprising compound, such as comprising 3-20 sulphates, preferably containing 4-15 sulphates, more preferably containing 5-12 sulphates, such as 6-10 sulphates, e.g. 7-8 sulphates, such as (C₅H₆XᵢO₁₀S₂)ₙ, wherein X may be selected from single valence metal atoms, such as Na⁺, and K⁺, i being 2, and from double valence metal atoms, such as Ca⁺, and Mg⁺, i being 1, and n preferably being in the range of 2-20, more preferably 3-12, such as 4-8. When reference to the first compound is made the references includes the oligo- and polysulfates, as indicated above. The molecular weight of the first compound is preferably not too large, such as <30 kDa, preferably <20 kDa, more preferably <10 kDa, even more preferably <7 kDa or even more <4.1 kDa, such as <2.7 kDa. A compound that can activate PPAR means that the PPAR receptor activity increases by at 2 to 3 times compared to a baseline situation without the PPAR activating compound. The compound that can activate PPAR is not a protein, or not a nucleic acid (NA)-strand compound. Suitable compounds that can activate PPAR include thiazolidinediones, NSAIDs, sulphonylureas and indoles. Inventors discovered that virus induced infections and disorders can be treated by a compound according to the invention, comprising a carbohydrate comprising more than one sulphate group like Pentosan polysulfate in combination with a PPAR, preferably a PPARγ agonist, like indomethacin or pioglitazone. It is noted that various documents refer to first and second compounds according to the invention individually, but not in combination. The synergistic effect of the present invention could therefore not observed. The synergistic effect is for instance found in a lower combined dosage, compared to the sum of the two individual dosages, that is required to find the same effect, typically a 2-10 times lower dosage, which lower dosage e.g. mitigates toxic side effects, the increased activity of the at least one second compound induced by the at least one first compound, and the decrease of side effects per se, in particular of the at least second compound. For instance, pentosan polysulfate may be used for reduction of alfa virus induced inflammation. Sulfated polysaccharides may inhibit action of various enveloped viruses. PPARγ activation may be used as an anti-inflammatory therapy for virus infections. Indomethacin may be used for antiviral activity against SARS virus. The synergistic effect of the present first and second compound is however not recited. The invention therefore provides a pharmaceutical composition, comprising a first compound comprising at least one carbohydrate (saccharide) and containing at least one sulphate, in combination with a second compound that can activate PPAR, preferably PPARγ, for use in the treatment of enveloped DNA or RNA virus induced infections and disorders.

Hence, patients with virus infections of enveloped DNA or RNA virus induced infections and disorders, benefit from use of the pharmaceutical composition of the invention.

Therefore, the invention also relates to the use of the pharmaceutical composition as described as a medicament.

Preferably, the invention further relates to the pharmaceutical composition of the invention, for use a medicament for use in the treatment of enveloped DNA or RNA virus induced infections and disorders.

In a second aspect the present invention relates to a dosage comprising separate dosage forms for individual pharmaceutical active ingredients, and/or wherein the composition is in the form of a tablet, capsule, repository, nanoparticles, or injectable.

### DETAILED DESCRIPTION OF THE INVENTION

In an exemplary embodiment the present pharmaceutical composition may comprise pentosan sulphate, and/or adequan sulphate.

In an exemplary embodiment of the present pharmaceutical composition for use in the treatment of enveloped DNA or RNA virus induced infections and disorders the virus may be selected from viruses with a positive-sense single-stranded RNA genome, such as of the Realm Ribovira, preferably of the order of Nidovirales or Articulavirales, more preferably of the family of Coronaviridae or Orthomyxoviridae or Flaviviridae, such as of the subfamily of Orthocoronavirinae.

In an exemplary embodiment of the present pharmaceutical composition for use in the treatment of enveloped DNA or RNA virus induced infections and disorders the first compound may be selected from pentosan polysulfate (CAS 37300-21-3 N or 116001-96-8, (C₅H₆Na₂O₁₀S₂)ₙ, n=1-10), Polysulfated glycosaminoglycan (adequan sulphate, dextran sulphate (CAS 9011-18-1), fucoidan (CAS 9072-19-9), and combinations thereof.

In an exemplary embodiment of the present pharmaceutical composition for use in the treatment of enveloped DNA or RNA virus induced infections and disorders the second compound may be selected from indomethacin (CAS 53-86-1), pioglitazone (CAS 112529-15-4), and combinations thereof.

In an exemplary embodiment of the present pharmaceutical composition for use in the treatment of enveloped DNA or RNA virus induced infections and disorders the first compound and second compound may be provided in a molar ratio of 0.01:1 to 1:0.01.

In an exemplary embodiment the present pharmaceutical composition for use in the treatment of enveloped DNA or RNA virus induced infections and disorders the composition may further comprise (iii) at least one pharmaceutically acceptable carrier.

In an exemplary embodiment of the present pharmaceutical composition for use in the treatment of enveloped DNA or RNA virus induced infections and disorders the active pharmaceutical ingredients may be in one dosage form, preferably comprising 1-10 mg active ingredients/kg body weight, such as 2-1000 mg active ingredients.

In an exemplary embodiment the present pharmaceutical composition for use in the treatment of enveloped DNA or RNA virus induced infections and disorders may comprise pentosan sulphate and/or adequan sulphate, and indomethacin and/or pioglitazone.

In an exemplary embodiment the present pharmaceutical composition for use in the treatment of enveloped DNA or RNA virus induced infections and disorders the pharmaceutical composition may comprise separate dosage forms for individual pharmaceutical active ingredients.

In an exemplary embodiment of the present pharmaceutical composition for use in the treatment of enveloped DNA or RNA virus induced infections and disorders the composition may be in the form of a tablet, capsule, repository, nanoparticles, or injectable.

In an exemplary embodiment of the present pharmaceutical composition for use in the treatment of enveloped DNA or RNA virus induced infections and disorders the composition may be in the form of a tablet or capsule suitable for oral administration.

In an exemplary embodiment of the present pharmaceutical composition for use in the treatment of enveloped DNA or RNA virus induced infections and disorders, wherein the use is for obese human beings. It is noted that obese people suffered more than less obese people, especially in terms of mortality. It has been found that the present composition functions especially well for obese people.

In an exemplary embodiment of the present pharmaceutical composition for use in the treatment of enveloped DNA or RNA virus induced infections and disorders, wherein the use is for recovery of lung infection, such as of lung fibrosis. It has been found that the present composition supports recovery.

In an exemplary embodiment of the present pharmaceutical composition for use in the treatment of enveloped DNA or RNA virus induced infections and disorders the disorder may be selected from respiratory tract infection, such as sarcoidosis, and pulmonary fibrosis.

In an exemplary embodiment of the present pharmaceutical composition for use in the treatment of enveloped DNA or RNA virus induced infections and disorders may be for use as a medicament by administering said medicament in an effective amount for a sufficient period.

In an exemplary embodiment of the present pharmaceutical composition for use in the treatment of enveloped DNA or RNA virus induced infections and disorders the administration may be to a pet or mammal comprising said virus infection.

In an exemplary embodiment of the present pharmaceutical composition for use in the treatment of enveloped DNA or RNA virus induced infections and disorders may be for use in the treatment of enveloped DNA or RNA virus induced infections and disorders.

The pharmaceutical composition according the invention may be a combination composition, wherein the active pharmaceutical ingredients are in one dosage form. The pharmaceutical composition may also comprise separate dosage forms for individual pharmaceutical active ingredients.

In an exemplary embodiment of the present pharmaceutical composition the active pharmaceutical ingredients may be in one dosage form, preferably comprising 1-10 mg active ingredients/kg body weight, such as 2-1000 mg active ingredients.

In an exemplary embodiment the present dosage is for a use after chronic inflammation occurred, such as after 7 days after inducement of the infection or disorder.

In an exemplary embodiment of the present dosage the dosage is provided during a fibrosis phase.

In an exemplary embodiment of the present dosage the dosage is for sub-cutaneous application, such as a weekly application, wherein a sub-cutaneous dosage comprises 20-100 mg active ingredient per dosage.

In an exemplary embodiment of the present dosage the at least one first compound and at least one second compound are provided in a weight ratio of 1:1 to 10:1, preferably in a weight ratio of 1.5:1 to 5:1, and wherein a total weight of active ingredients is from 1-100 mg per dosage.

The compound that activates PPAR, preferably PPARγpreferably is a compound that can activate PPAR, meaning the PPAR receptor activity increases by at least 2-3 times compared to a baseline situation without the PPAR activating compound.

The compound that activates PPAR, preferably PPARγ preferably has anti-inflammatory effects, meaning that at least one of the inflammatory markers is reduced. Inflammatory markers are e.g. TNFα, IFN, cytokines, histamine, interleukins, chemokines, leukotrienes, lysosome granules and prostaglandins.

A carbohydrate is an organic compound comprising only carbon, hydrogen, and oxygen, usually with a hydrogen:oxygen atom ratio of 2:1 (as in water); with the empirical formula Cₘ(H₂O)ₙ (where m could be different from n). Structurally it is more accurate to view carbohydrates as polyhydroxy aldehydes and ketones.

Carbohydrates as used in the present invention do not relate to glycosylated proteins or nucleoside compounds (like DNA, RNA or the like)

The carbohydrate preferably is a Glycosaminoglycans (GAGs) or keratan. Glycosaminoglycans, or mucopolysaccharides are long unbranched polysaccharides consisting of a repeating disaccharide unit. The repeating unit consists of an amino sugar (N-acetylglucosamine or N-acetyl galactosamine) along with a uronic sugar (glucuronic acid or iduronic acid) or galactose.

Based on core disaccharide structures, GAGs may be classified into four groups.

Preferred types of sulphated saccharides are also known as heparin, heparan sulphate analogues, or heparin-like compounds.

Suitable compounds comprising a carbohydrate and a sulphate group can be derived from natural sources, or can be made—at least in part—synthetically.

Examples of sulphated compounds include single Sulphur containing agents like MSM (dimethylsulfon), dextran sulphate, or multiple Sulphur containing agents, like polysulfated glycosaminoglycan (adequan sulphate), heparin like pentosan sulphate or the like.

Preferred sulphated saccharides compounds are polysulfated glycosaminoglycan (Adequan sulphate), heparin like pentosan polysulfate (Elmiron) or the like.

The second part of the present composition, is an agent that can activate PPAR, preferably PPARγ. Preferred PPAR agonists include triglitazones (TZDs: triglitazone, rosiglitazone, pioglitazone etc.) and indomethacin.

Preferably, both the first compound comprising at least one carbohydrate and containing at least one sulphate and the second compound that activates PPARγ have a combined, synergistic effect.

Whether a compound is a PPAR, preferably PPARγ agonist can be determined in a simple cell-based test.

By PPAR agonist is meant any compound that increases the biological activity or expression of one or more PPARs (e. g., PPARα, PPARγ, and PPARβ/δ) in a cell by a least 10% relative.

Examples of PPARγ agonists include any of the Thiazolidinediones, but particularly Rosiglitazone, Troglitazone and Pioglitazone and analogs thereof. Rosiglitazone is the preferred PPAR agonist. Additional examples of PPARγ agonists include non-steroidal anti-inflammatory drugs, such as Indomethacin, Ibuprofen, Naprosyn and Fenoprofen and antioxidants such as vitamin E, vitamin C, S-adenosyl methionine, selenium, beta-carotene, idebenone, cysteine, dithioerythritol, dithionite, dithiothreitol, and pyrosulfite.

Examples of PPARα agonists include any of the fibrates (e.g., fenofibrate, bezafibrate, gemfibrozil, and analogs thereof), docosahexaenoic acid, and Wy 4643.

So, for example Pentosan sulphate in combination with one of these compounds could be used in the present treatment. More specifically PPARγ agonistic action can be determined in a similar manner as described above, using a dual luciferase assay with a luciferase construct containing a PPARγ responsive element.

This second compound is not a protein or nucleic acid-based compound.

Suitable PPARγ agonists include thiazolidinediones, NSAIDs, sulphonyl ureas and indoles.

Thiazolidinediones (abbreviated as TZDs) are also known as glitazones. Suitable thiazolidinediones include rosiglitazone, pioglitazone, troglitazone, and ciglitazone, which are selective ligands for the nuclear transcription factor peroxisome proliferator-activated receptor (PPAR)y. Typical TZDs have the formula as shown in FIG. 1, where n is 1,2, or 3,Y and Z independently are O or NH; and E is a cyclic or bicyclic aromatic or non-aromatic ring, optionally containing a heteroatom selected from oxygen or nitrogen. Suitable TZDs are for example described in WO2000/27401.

NSAIDs are a class of drugs that provides analgesic and antipyretic (fever-reducing) effects, and, in higher doses, anti-inflammatory effects. PPARγ is activated by several endogenous ligands emerging from the metabolism of arachidonic acid and linoleic acid. Among the PPARγ ligands represented are the lipoxygenase products 13(S)HODE (produced from linoleic acid by 15-LOX-1) and 15(S)HETE (produced from arachidonic acid by both 15-LOX-1 and 15-LOX-2, although 15-LOX-2 catalyzes this reaction much more efficiently). Induction of 15-LOX-1 activity by NSAIDs occurs independently of COX-2 inhibition

NSAIDs can be classified based on their chemical structure or mechanism of action. Older NSAIDs were known long before their mechanism of action was elucidated and were for this reason classified by chemical structure or origin. Newer substances are more often classified by mechanism of action.

Suitable NSAIDs include salicylates, propionic acid derivatives, acetic acid derivatives, enolic acid (oxicam) derivatives and fenamic acid derivatives,

Suitable salicylates include aspirin (acetylsalicylic acid), diflunisal (Dolobid™), salsalate (Disalcid™) and choline magnesium trisalicylate (Trilisate™)

Suitable propionic acid derivatives include ibuprofen, dexibuprofen, naproxen, fenoprofen, ketoprofen, dexketoprofen, flurbiprofen, oxaprozin, and loxoprofen.

Suitable acetic acid derivatives include Indomethacin, tolmetin, sulindac, etodolac, ketorolac, diclofenac and nabumetone (drug itself is non-acidic but the active, principal metabolite has a carboxylic acid group)

Suitable enolic acid (oxicam) derivatives include piroxicam, meloxicam, tenoxicam, droxicam, lornoxicam, and isoxicam.

Suitable fenamic acid derivatives (fenamates) include mefenamic acid, meclofenamic acid, flufenamic acid, and tolfenamic acid.

Sulfonylurea derivatives are a class of antidiabetic drugs that are used in the management of diabetes mellitus type 2. Examples include carbutamide, acetohexamide, chlorpropamide, tolbutamide, tolazamide, glipizide, gliclazide, glibenclamide (glyburide), glibornuride, gliquidone, glisoxepide, glyclopyramide and glimepiride. They act primarily by increasing insulin release from the beta cells in the pancreas. All sulfonylureas contain a central S-phenyl sulfonylurea structure with a p-substituent on the phenyl ring (R) and various groups terminating the urea N′ end group (R2) (see FIG. 2).

Indoles include indole-derived agents which can bind to PPARγ, and comprise mainly sulfonyl-indoles.

Dual alpha-gamma agonists are suitable as well, and include glitazars.

Suitable glitazars include aleglitazar, muraglitazar and tesaglitazar.

PPARα agonists include fibrates and biguadines.

Preferably, the compound comprising at least one saccharide and at least one sulphate can be present in the pharmaceutical composition such as in an amount of 0-10 wt. % relative to a total weight.

Preferably, the PPARγ agonist can be present in an amount of 0-10 wt. % relative to a total weight.

Preferably, the relative amounts between the two compounds (sulphated compound to PPARγ agonist) are 1:1 to 1:5.

The pharmaceutically acceptable carrier can be present in an amount of 0.01% to 99.9%, preferably 0.1%-10%, and its amount will depend on the formulation.

In one embodiment, the medicament is in the form of a tablet, suitable for oral administration.

In another embodiment, the medicament is in a form suitable for local administration.

The medicament can comprise the components in the form of a solid, or liquid preparation.

The dosage form can be an immediate release or extended release formulation.

It is an advantage of the present invention that the constituents of the composition can be non-biologics, and several of the exemplary components are approved medicaments.

Pentosan polysulphate (PPS), manufactured from beech-wood, is an FDA-approved oral medication for the treatment of interstitial cystitis (IC), also known as painful bladder syndrome. PPS is known to have anti-inflammatory and pro-chondrogenic properties. Pentosan polysulphate is available as pills or as a direct infusion into the bladder.

Adequan sulphate, a polysulfated glycosaminoglycan, is a well-known veterinary medicament for treating joint pain.

Indomethacin is an FDA-approved, non-steroidal anti-inflammatory drug (NSAID). It's commonly used to reduce fever, pain, stiffness and swelling. Furthermore, Indomethacin is a COX-inhibitor that blocks prostaglandin production and is used as an inhibitor of inflammation. In higher doses it can also induce adipogenesis in vitro in mesenchymal progenitor cells by activating PPARγ.

Pioglitazone is a powerful PPARγ agonist, and belongs to the class of thiazolidinediones, or glitazones, and was designed to treat type II diabetes by increasing the insulin sensitivity.

### SUMMARY OF THE FIGURES

FIG. 1 shows a schematic structure of Thiazolidinedione and FIG. 2 of sulfonylurea.

FIG. 3 shows Polysulfated glycosaminoglycan.

FIG. 4 shows schematics of bleomycin induced fibrosis and FIG. 5 effects thereof on lung tissue, showing H&E and SMA staining.

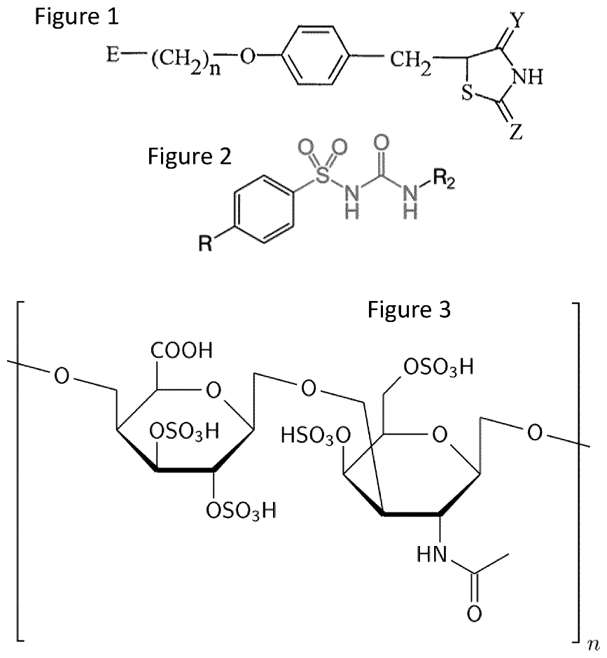

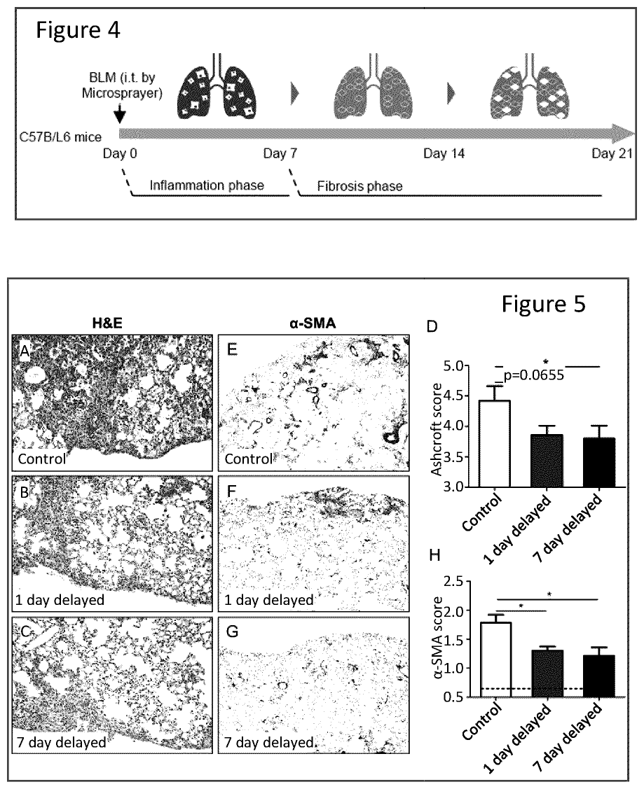

### EXPERIMENTAL SET-UP

The inducement of fibrosis and the mouse model used largely follow the procedure as described in Ruscitti et al. Multi-disciplinary Respiratory Medicine (2017) 12:8, DOI 10.1186/s40248-017-0089-0.

The present first and second compound, such as pentosan, is typically provided in the drinking water, such as at a dosage of 20-25 mg/kg/day, and/or sub-cutaneous of e.g. 50 mg/kg/week.

Experiment:

Day 1 baseline blood draws and intra tracheal injection of 80 μl of physiological saline (control for bleomycin) with Isoflurane anesthesia.

Day 7 baseline blood draws

Day 7 start treating with compounds (n=28, n=4 per group)

1 compound B (second compound, e.g. indomethacin) 4 mg/kg (DMSO and trapsol) drinking water

2 compound C (alternative/additional second compound) 40 mg/kg (DMSO and trapsol) drinking water

3 compound A (first compound) 20 mg/kg and B 4 mg/kg (DMSO and trapsol) drinking water

4 compound A 20 mg/kg and C 40 mg/kg (DMSO and trapsol) drinking water

5 compound A 50 mg/kg/week subcutaneously weekly and B 4 mg/kg (DMSO and trapsol) drinking water

6 compound A 50 mg/kg/week subcutaneously weekly and C 40 mg/kg (DMSO and trapsol) drinking water

7 control (DMSO and trapsol) saline subcutaneous weekly+drinking water (added because we need a control group without compounds to compare with given FACS BAL dif staining and activation markers on the alveolar macrophages)

Day 14 change drinking water and subcutaneous injections with A

Day 21 sacrifice mice.

Collection of:

Blood divided into 3 portions (Acrp30 (5 μl), cytokines (50 μl) and remainder); measure at regular intervals, typically 5 times, whereof 2 times after treatment starts; measure e.g. adiponectin levels. Adipocyte complement-related protein of 30 kDa (Acrp30, adiponectin, or AdipoQ) is a fat-derived secreted protein that circulates in plasma. Acrp30 is lower in insulin-resistant states and it is implicated in the regulation of in vivo insulin sensitivity. Plasma Acrp30 levels from two diabetic mouse models were increased in response to treatment.

Results suggest that induction of Acrp30 may represent a key mechanism that contributes to the beneficial metabolic effects of the present compounds and that measurement of Acrp30 levels prove to be a valuable biomarker that can be used to gauge the extent of in vivo activation.

FACS analysis (fluorescence activated cell sorting) of cells in BAL (bronchoalveolar lavage), a ELISA (enzyme-linked immunosorbent assay) in bronchoalveolar fluid (BALF).

Lung histology (freeze) and left lobe for hydroxyproline assay.

Results

From the experiments it follows that first of all a positive effect was observed in the treatment of (bleomycin) induced fibrosis. In addition, the combined pharmaceutical composition of the present invention showed a synergistic effect over the individually applied first and second compounds, respectively. The synergistic effect of the present composition was typically at least 2 times the summed individual effects, mostly at least 10 times, and often at least 20 times, hence at least a factor higher. Also, (bleomycin-induced) fibrosis was largely or fully mitigated, that is e.g. lung tissue preserved at least partly.

1. A pharmaceutical composition for use in the treatment of enveloped DNA or RNA virus induced infections and disorders, respiratory tract infections, and lung disorders, comprising (i) at least one first compound comprising at least one carbohydrate and containing more than one sulphate selected from multiple Sulphur containing agents with a molecular weight of <30 kDa, in combination with (ii) at least one second compound selected from the group of non-protein and non-NA-strand compounds, different from (i), that can activate PPAR,
wherein the at least one first compound and at least one second compound are provided in a molar ratio of 0.01:1 to 1:0.01.

2. The pharmaceutical composition for use in the treatment of enveloped DNA or RNA virus induced infections and disorders according to claim 1, wherein the use is selected from a use for recovery of lung infection, for recovery of lung disorders, and for recovery of lung fibrosis.

3. The pharmaceutical composition for use in the treatment of enveloped DNA or RNA virus induced infections and disorders according to claim 1, wherein the virus is selected from viruses with a positive-sense single-stranded RNA genome.

4. The pharmaceutical composition for use in the treatment of enveloped DNA or RNA virus induced infections and disorders according to claim 1, , wherein the first compound is selected from pentosan polysulfate (CAS 37300-21-3 N or 116001-96-8, (C₅H₆Na₂O₁₀S₂)ₙ, n=1-10), Polysulfated glycosaminoglycan, dextran sulphate (CAS 9011-18-1), fucoidan (CAS 9072-19-9), and combinations thereof.

5. The pharmaceutical composition for use in the treatment of enveloped DNA or RNA virus induced infections and disorders according to claim 1, wherein the second compound is selected from indomethacin (CAS 53-86-1), pioglitazone (CAS 112529-15-4), and combinations thereof.

6. The pharmaceutical composition for use in the treatment of enveloped DNA or RNA virus induced infections and disorders according to claim 1, wherein the composition further comprises (iii) at least one pharmaceutically acceptable carrier.

7. The pharmaceutical composition for use in the treatment of enveloped DNA or RNA virus induced infections and disorders according to claim 1, selected from a pharmaceutical composition comprising pentosan sulphate and/or adequan sulphate, and from a pharmaceutical composition comprising indomethacin and/or pioglitazone.

8. The pharmaceutical composition for use in the treatment of enveloped DNA or RNA virus induced infections and disorders according to claim 1, wherein the use is for obese human beings.

9. The pharmaceutical composition for use in the treatment of enveloped DNA or RNA virus induced infections and disorders according to claim 1, for use as a medicament by administering said medicament in an effective amount for a sufficient period.

10. The pharmaceutical composition for use in the treatment of enveloped DNA or RNA virus induced infections and disorders of claim 9, wherein the administration is selected from the administration to a pet and from the administration to a mammal, comprising said virus infection.

11. The pharmaceutical composition according to claim 1 as a medicament selected from the use in the treatment of enveloped DNA or RNA virus induced infections and disorders, and the use in the treatment of fibrosis.

12. The pharmaceutical composition for use in the treatment of enveloped DNA or RNA virus induced infections and disorders according claim 1, wherein the active pharmaceutical ingredients are in one dosage form, comprising 1-10 mg active ingredients/kg body weight.

13. A dosage according to the dosage of claim 12, comprising separate dosage forms for individual pharmaceutical active ingredients,
and wherein the composition is in the form selected from a tablet, a capsule, a repository, nanoparticles, and an injectable.

14. The dosage according to claim 12, for a use after chronic inflammation occurred, and/or
wherein the dosage is provided during a fibrosis phase, and
wherein the dosage is for sub-cutaneous application, wherein a sub-cutaneous dosage comprises 20-100 mg active ingredient per dosage.

15. The dosage according to claim 12, wherein the at least one first compound and at least one second compound are provided in a weight ratio of 1:1 to 10:1, and wherein a total weight of active ingredients is from 1-100 mg per dosage.

16. The pharmaceutical composition for use in the treatment of enveloped DNA or RNA virus induced infections and disorders according to claim 1, wherein the at least one second compound is selected from PPARγ, thiazolidinediones, NSAIDs, sulphonylureas, and indoles, and a salt thereof.

17. A method for treating enveloped DNA or RNA virus induced infections and disorders, respiratory tract infections, or lung disorders in a subject in need thereof, wherein the method comprises administering to the subject a therapeutically effective amount of the pharmaceutical composition of claim 1.

18. The method of claim 17 wherein the virus is selected from viruses with a positive-sense single-stranded RNA genome.
